(19) United States
(12) Patent Application Publication
(10) Pub. No.: US 2010/0036140 A1
(43) Pub. Date: Feb. 11, 2010

(54) STEROLS MODIFIED BY POLYETHYLENE GLYCOL, THE PREPARATION AND THE USE THEREOF

(76) Inventor: Wenfang Zhang, Beijng (CN)

Correspondence Address: AUSTIN RAPP & HARDMAN, 170 South Main Street, Suite 735, SALT LAKE CITY, UT, 84101, US

(21) Appl. No.: 12/519,692

(22) PCT Filed: Dec. 13, 2007
(86) PCT No.: PCT/CN07/03585

§ 371 Date: Jun. 17, 2009

(30) Foreign Application Priority Data
Dec. 18, 2006 (CN) 200610168130.1

(51) Int. Cl. C07J 9/00; C07C 401/00

(52) U.S. Cl. 552/547; 552/653

(57) ABSTRACT

The invention provides sterols modified by polyethylene glycol represented by the following formula, the preparation and the use thereof, wherein each symbol is defined as in the description.

### FIELD OF THE INVENTION

The invention relates to a kind of sterol compound (PGC) modified by polyethylene glycol (PEG), the preparation and the use thereof.

### BACKGROUND OF THE INVENTION

Sterol is a kind of perhydrocyclopentanophenanthrene derivatives. Sterol compound comprises cholesterol, 7-dehydrocholesterol, ergosterol, vitamin D3, vitamin D2, and etc.

Polyethylene glycol is a condensed product of ethylene glycol, which has good amphiphilic property and is generally used as a modifier because of its low toxicity and non-antigenic (its biocompatibility has been approbated by U.S. FDA). For example, polyethylene glycol is generally used to modify phosphatidylcholine and fatty acids, and the product thereof can be used as an emulsion or liposome excipient. Polyethylene glycol is also used to modify proteins (also called PEGylation of proteins) including modifying the physics conjugates and chemically modified materials of PEG, protein and polypeptide drugs. PEGylation of drugs can increase the solubility of the drugs, reduce the immunogenicity and the eliminate rate, increase the therapeutic index of protein drugs, and expand the clinical application.

China Invention Patent Application No. 00110157.9 discloses a compound comprising polyethylene glycol and cholesterol which connected by succinic acid through ester linkage. The compound is unsuitably used as an injection excipient due to the strong hemolytic property while injecting.

### SUMMARY OF THE INVENTION

After a large number of researches, the inventor of the present invention found that sterol modified by polyethylene glycol which is connected to sterol by small molecule compounds through ether linkage has a higher safety, and can be used as an injection excipient, so that the drug loading capacity thereof is substantially increased, while the targeting distribution and the slow release of the drugs in vivo can be carried out.

Therefore, the present invention provides a kind of sterol modified by polyethylene glycol, wherein the formula thereof is

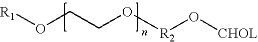

wherein CHOL is a sterol compound;

R₁ is H, straight or branched alkyl, alkenyl or alkynyl, or straight or branched alcohol, ketone, ether, ester, carboxyl or salt thereof, amine or amide group;

R₂ is straight or branched alkyl, alkenyl or alkynyl, or ketone, ether, ester, amine or amide group;

n is an integer from 1 to 40.

The present invention also provides a method for preparing said sterol modified by polyethylene glycol, comprising:

(a) synthesizing a steryl p-toluenesulfonate;
(b) reacting steryl p-toluenesulfonate synthesized by step (a) with a kind of OH—R₂—OH;

(c) reacting a product of step (b) with a kind of polyethylene glycol;

(d) optionally, introducing one R₁ group into a product of step (c).

The present invention also provides a use of said sterol, said sterol is used as a surfactant for manufacturing aqueous dispersion system, such as nano-emulsion, liposome; also used as one component of a carrier for manufacturing micelle or solid lipid nanoparticles which can be dispersed in water; also used as one component of a long-circulation nano-carrier component, temperature-sensitive or PH-sensitive nano-carrier component; also used as a cosmetic excipient which has very good moisturizing effect; and applied to hypodermic for smoothing away wrinkles.

### BRIEF DESCRIPTION OF THE DRAWINGS

FIG. 1 is a mass spectrogram of condensed product of butylene glycol and cholesterol.

FIG. 2 is an infrared spectrum of a kind of sterol modified by polyethylene glycol according to the present invention.

FIGS. 3A and 3B are mass spectrograms of a kind of sterol modified by polyethylene glycol according to the present invention.

FIG. 4 is an infrared spectrum of another kind of sterol modified by polyethylene glycol according to the present invention.

FIGS. 5A and 5B are mass spectrograms of another kind of sterol modified by polyethylene glycol according to the present invention.

FIG. 6 is an average plasma drug concentration-time curve of voriconazole after the intravenous injection of two kinds of 36 mg/kg voriconazole in rats.

### DESCRIPTION OF THE INVENTION

The present invention provides a sterol modified by polyethylene glycol, wherein the formula thereof is

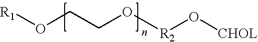

wherein CHOL is a sterol compound;

R₁ is H, straight or branched alkyl, alkenyl or alkynyl, or straight or branched alcohol, ketone, ether, ester, carboxyl or salt thereof, amine or amide group;

R₂ is straight or branched alkyl, alkenyl or alkynyl, or ketone, ether, ester, amine or amide group;

n is an integer from 1 to 40.

In a preferred embodiment of the present invention, CHOL is cholesterol, 7-dehydrocholesterol, ergosterol, vitamin D3 or vitamin D2.

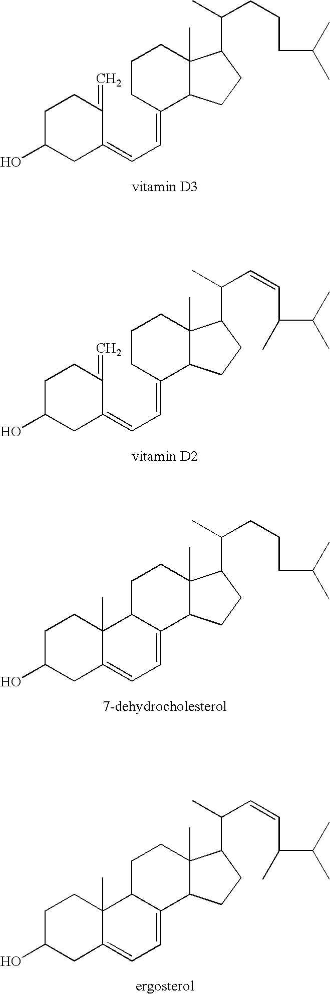

In a further preferred embodiment of the present invention, CHOL is cholesterol.

In a preferred embodiment of the present invention, R₁ is H, —CH₃, —CH₂CH₃, n-propyl or isopropyl, n-butyl, isobutyl, sec-butyl, tert-butyl, —CH₂CH(CH₂CH₂)ₘCH₃, —CH═CHCH₃, —CH₂C═CH₂, —CH₂OH, —CH₂COCH₃, —CH₂O(CH₂CH₂)ₘCH₃, —CH₂COOH, —CH₂CH₂NH₂ or —COCH₃, wherein m is an integer from 1 to 30.

In a preferred embodiment of the present invention, R₂ is —CH₂—, —CH₂(CH₂)ₘCH₂— —CH₂C(CH₃)₂—CH₂—(CH₂)ₘ, —CH₂CH₂(CH₂CH₂)ₘCH₂—, —CH═CHCH₂—, —CH₂C═CH—, —CH₂OCH₂—, —CH₂COCH₂—, —CH₂O(CH₂CH₂)ₘCH₂—, —CH₂COOCH₂—, m is an integer from 1 to 30, wherein each of said groups is optionally substituted by one or more amino group.

In a further preferred embodiment of the present invention, R₁ is —H, —CH₃, or —COCH₃.

In a further preferred embodiment of the present invention, R₂ is —CH₂CH₂—, —CH₂(CH₂)ₘCH₂—, —CH₂C(CH₃)₂—CH₂—(CH₂)ₘ—, m is an integer from 1 to 12, preferably from 1 to 8, especially from 1 to 6, wherein each of said groups is optionally substituted by one or more amino group.

In a preferred embodiment of the present invention, n in the formula of sterol modified by polyethylene glycol is from 3 to 20, preferably from 5 to 20.

The method for preparing said sterol of the present invention comprises:

(a) synthesizing a steryl p-toluenesulfonate;

(b) reacting steryl p-toluenesulfonate synthesized by step (a) with a kind of OH—R₂—OH;

(c) reacting a product of step (b) with a kind of polyethylene glycol;

(d) optionally, introducing one R₁ group into a product of step (c).

In the step (a) of the method of the present invention, sterol is preferably reacted with p-toluene sulfonyl halide to form steryl p-toluenesulfonate, wherein the halide is preferably fluoride, chloride or bromide, especially chloride.

Certainly, commercially available steryl p-toluenesulfonate can be directly used in the step (b) of the method of the present invention.

In the step (b) of the method of the present invention, the definition, preferred definition and further preferred definition of used HO—R₂—OH is as mentioned above.

In the step (c) of the method of the present invention, product of the step (b) is preferably activated by p-toluene sulfonyl halide (wherein the halide is preferably fluoride, chloride or bromide, especially chloride), and then reacted with polyethylene glycol, wherein solvent is preferably 1,4-dioxane.

Optionally (i.e. selectively but not obligatory), continuing to implement the step (d) of the method of the present invention after the step (c), wherein the product of the step (c) is reacted with compound of R₁—OH, R₁—X or R₁—(C=0)X, preferably one of alkylation reaction, esterification reaction and etc, wherein the definition, preferred definition and further preferred definition of R₁ is as mentioned above, X is halide, preferably fluoride, chloride or bromide, especially chloride.

In a preferred embodiment of the method of the present invention, sterol is cholesterol, 7-dehydrocholesterol, ergosterol, vitamin D3 or vitamin D2, especially cholesterol.

In a preferred embodiment of the method of the present invention, HO—R₂—OH is preferably HO—CH₂CH₂—OH, HO—CH₂(CH₂)ₘCH₂—OH, HO—CH₂C(CH₃)₂—CH₂—(CH₂)ₘ—OH, wherein m is an integer from 1 to 30, preferably from 1 to 12, especially from 1 to 8, particularly from 1 to 6, wherein each group represented by R₂ is optionally substituted by one or more amino group.

In the method of the present invention, p-toluene sulfonyl halide preferred used in the step (a) and the step (c) can be the same, also can be different; preferably, the same p-toluene sulfonyl halide between the two steps, especially p-toluene sulfonyl chloride, is used.

The method of the present invention is implemented in the temperature which is lower than the decomposition temperature of the reaction system, and appropriate choices can be specifically made according to the composition of the reaction system.

The method of the present invention is implemented in the atmospheric pressure; but can also be implemented in an increased pressure.

In the step (c) and the step (d) of the method of the present invention, the number of repeat units (that is the n value in (CH₂—CH₂—O)ₙ) of polyethylene glycol in the obtained product is from 1 to 40, preferably from 3 to 20, and especially from 5 to 20.

It can be understood by the man skilled in the art that solvent can be used if necessary in the present invention, and the person having ordinary skill in the art can make a reasonable choice according to the contents disclosed by this specification.

In a specific embodiment, the method of the present invention is implemented by the following steps:

a) Under the protection of nitrogen, dissolving sterol in anhydrous pyridine, adding p-toluene sulfonyl chloride followed by stirring under room temperature, pouring in ice water after finishing the reaction, filtering to collect solid, washing till to neutral, and drying to gain a white solid which is steryl p-toluenesulfonate;

b) Under the protection of nitrogen, dissolving steryl p-toluenesulfonate in HO—R₂—OH and 1,4-dioxane, heating and stirring to react 2 to 4 hours, removing dioxane after finishing the reaction, adding residues into water, extracting with ether for two or three times, then washing with 5% to 10% sodium carbonate solution once, washing with water once, drying with anhydrous magnesium sulfate, and removing solvent to gain crude product, wherein column chromatography of the crude product is carried out to gain a light yellow solid M1;

c) Under the protection of nitrogen, dissolving M1 in anhydrous pyridine, adding p-toluene sulfonyl chloride into a reaction bottle, stirring under room temperature, pouring in ice water after finishing the reaction, filtering to collect solid, washing to neutral, and drying to gain a light yellow solid M2; under the protection of nitrogen, dissolving M2 in polyethylene glycol and 1,4-dioxane, stirring to react for 2 hours at 80° C., removing dioxane after finishing the reaction, adding residues into water, extracting with ether twice, then washing with 5% to 10% sodium carbonate solution for one or two times, washing with water one or two times, drying with anhydrous magnesium sulfate, and removing solvent to gain crude product, wherein column chromatography of the crude product is carried out to gain the compound of the present invention: sterol ether modified by polyethylene glycol.

d) Furthermore, i) when R₁ is acetyl, dissolving said product of sterol ether modified by polyethylene glycol into solvent, adding acetyl chloride by dropping, pouring in water after finishing the reaction, washing with water till organic phase is into neutral, drying with anhydrous magnesium sulfate, and removing solvent to gain crude product, wherein column chromatography of the crude product is carried out to gain the compound of the present invention: sterol ether modified by 2-acetyl-polyethylene glycol.

ii) When R1 is methyl, dissolving said product of sterol ether modified by polyethylene glycol in water, adding dimethyl sulfate, adding alkaline (such as hydroxide of alkali metal or alkaline earth metal, for example sodium hydroxide) solution slowly by dropping, adjusting PH value as 2.0 after finishing the reaction, filtering by air pumping, and re-crystallizing the solid by silica gel column chromatography to gain the compound of the present invention: sterol ether modified by 2-methyl-polyethylene glycol.

The present invention provides the use of said sterol modified by polyethylene glycol, including said sterol used as carrier of hydrophobic drug or cosmetic excipient, or used to process drugs of hypodermic for smoothing away wrinkles.

Specifically, the sterol of the present invention can be used as carrier of hydrophobic drug, for example combining with hydrophobic drug to form nano-emulsion, liposome and micelle or nanoparticles which can be dispersed in water, wherein the amount of drugs is 0.001-30% of the total solids, the particle diameter distribution thereof is 10-1000 nm; also used as excipient of advanced cosmetic, wherein it has very good moisturizing effect and also can be applied to hypodermic for smoothing away wrinkles while used as a cosmetic excipient.

The drugs which can use the sterol of the present invention including, but not limited to, the following hydrophobic drugs: antibacterial drug, antiviral drug, antifungal drug, anti-inflammatory substance, coronary vasodilator, cerebral vasodilator, vasoconstrictor, quasi-psychotropic drug, anti-tumor drug, stimulating drug, anti-histamine drug, anti-hypertensive drug, vasoconstrictor drug, anti-migranine drug, anti-thrombotic drug, anti-arrhythmic drug, Vitamin, anti-diarrheal, analgesic, muscular nerve drug, reagent used in the central nervous system, and biological medicine including protein, polypeptide, peptide, and etc., which has poor solubility. More specifically, the drugs which can use the sterol of the present invention include Paclitaxel, Docetaxel, Ibuprofen, Adriamycin Series, Teniposide, Etoposide, Daunomycin, Mitomycin, Methotrexate, Mitomycin C, Indomethacin, Cyclosporine, Alprostadil, Propofol, Nimodipine, Vinorelbine, Hydroxycamptothecin, Cytarabine, Raltitrexed, Clarithromycin, Voriconazole, Itraconazole, Amphotericin B, Calcipotriol, Cisplatin, Oxaliplatin, Nedaplatin, and etc.

The preparation prepared by sterol modified by polyethylene glycol of the present invention and drug includes following route of administration: orally, injection, transdermal delivery or mucosa delivery, the preparation method thereof is as follows:

I. The Method for Preparing Nano-Emulsion by the Sterol of the Present Invention and Hydrophobic Drug

1. Inferior nano-emulsion of injection, the preparation method thereof is as follows:

a) dissolving drug in the appropriate amount of solvent, adding glyceride fatty acid, triglyceride, fatty acid, PGC (i.e. the sterol compound modified by polyethylene glycol) and phospholipid, heating to melt to oil phase, removing the volatile solvent, dissolving the appropriate amount of glycerol in proper water (PH 10-11) to form water phase at 50-90° C. with high-speed stirring, mixing oil phase with water phase at 50-90° C. with high-speed stirring to form pre-emulsion, and adjusting PH value to 4.5-8.0.

b) taking said pre-emulsion of the step (a), adjusting volume to predetermined recipe quantity with water of injection, transferring to a high-pressure emulsificating machine to emulsify repeatedly till the average particle diameter of emulsion drops is less than or equal to 5 um.

c) taking said emulsion of the step (b), bottling in a bottle filled with nitrogen after filtration, and sterilizing it to obtain the product.

2. nano-emulsion, the preparation method thereof is as follows:

a) dissolving drug in the appropriate amount of solvent, adding PGC, heating to melt, then adding recipe quantity of water (PH10-1) with completely stirring, adding proper assistant surfactant (ethanol or propylene glycol) to gain clear liquid or semi-clear liquid, and adjusting PH value to 4.0-9.0.

b) taking said emulsion of the step (a) to sterilize and filter, bottling in a bottle filled with nitrogen, and sterilizing it to obtain the product.

Oral emulsion can also contain aromatic and preservative, wherein the aromatic contains one or more than one of orange flavor, banana flavor, strawberry flavor and cream flavor, and preservative contains one or more than one of Nipagin esters and benzoic acid.

II. The Method for Preparing Micelle which Made from PGC and Hydrophobic Drug

a) dissolving hydrophobic drugs, PGC and appropriate stabilizer (such as block copolymer of poly-lactic acid and polyethylene glycol or copolymer of polyethylene oxide and poly-amino acid) in proper solvent, steaming to remove the solvent by decompression, adding recipe quantity of water, stirring uniformly, and high-speed stirring till the average particle diameter of emulsion drops is less than or equal to 5 um.

b) taking said emulsion, bottling in a bottle filled with nitrogen after filtration, and sterilizing it to obtain the product.

III. The Method for Preparing Liposome from PGC and Hydrophobic Drug

a) dissolving drug, PGC and lecithin in proper solvent, steaming to remove the solvent by decompression, adding recipe quantity of phosphate-buffered salt solution, and completely stirring to form multi-vesicular liposome.

b) taking said multi-vesicular liposome of the step (a), transferring to a high-pressure emulsificating machine to emulsify repeatedly till the average particle diameter of emulsions drops is less than or equal to 5 um.

c) taking said emulsion of the step (b), bottling in a bottle filled with nitrogen after filtration, and sterilizing it to obtain the product.

IV. The Method for Preparing Nanoparticles from PGC and Hydrophobic Drug

a) Heating to melt the recipe quantity of monostearin, fatty acid, lecithin, PGC and drug to form oil phase, adding frozen-dried supporting agent (preferably lactose, sucrose, maltose, mannitol and low-molecular weight L-dextran one or more than two) into recipe quantity of water (PH 10-11) of injection to form water phase, mixing oil phase and water phase by high-speed stirring to form pre-emulsion, adjusting PH value to 4.5-8.0, then repeating to high pressure homogenize and emulsify the pre-emulsion system by a high pressure homogenizer, until the particle diameter meet the required regulation.

b) Filtering said pre-emulsion, freezing to dry and sealed with filled nitrogen, so as to obtain the product.

The present invention will now be further described with reference to the following non-limiting examples:

### EXAMPLES

### Examples for Preparation

In each preparation examples, n in the structural formula of sterol modified by polyethylene glycol depends on the used polyethylene glycol; the n value showed in the bottom of the structural formula is a calculated value based on the used polyethylene glycol.

### Example 1 for Preparation

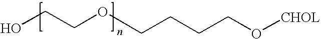

Wherein n is about 10.

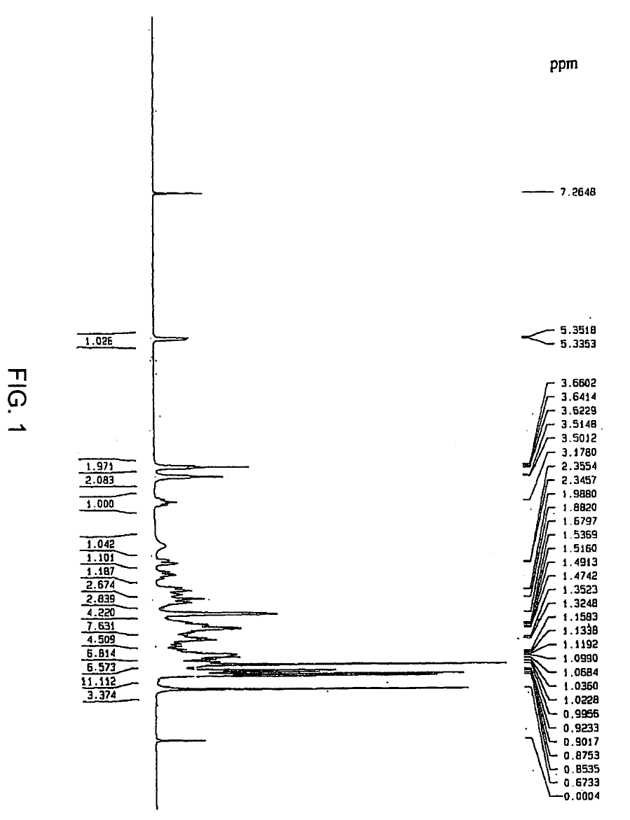

Under the protection of nitrogen, dissolving cholesterol (20 g) in anhydrous pyridine (100 ml), adding p-toluene sulfonyl chloride (9.8 g) into a reaction bottle followed by stirring under room temperature for 16 hours, TLC tracking till finishing the reaction, pouring in 200 ml ice water of 0° C., filtering to collect solid, washing till to neutral, and drying to gain 25 g white solid which is cholesterol p-toluenesulfonate; under the protection of nitrogen, dissolving cholesterol p-toluenesulfonate (21.74 g) in 200 ml 1,4-butanediol and 300 ml 1,4-dioxane, stirring to react for 2 hours at 80° C., TLC tracking till finishing the reaction, removing dioxane by decompression concentration, adding residues into 400 ml water, extracting with 200 ml ether twice, then washing with 100 ml 5% to 10% sodium carbonate solution for one or two times, washing with water (100 ml) one or two times, drying with anhydrous magnesium sulfate, and removing solvent to gain crude product, wherein column chromatography of the crude product is carried out to gain the pure: a light yellow solid M1-1, the mass spectrogram thereof is showed in FIG. 1.

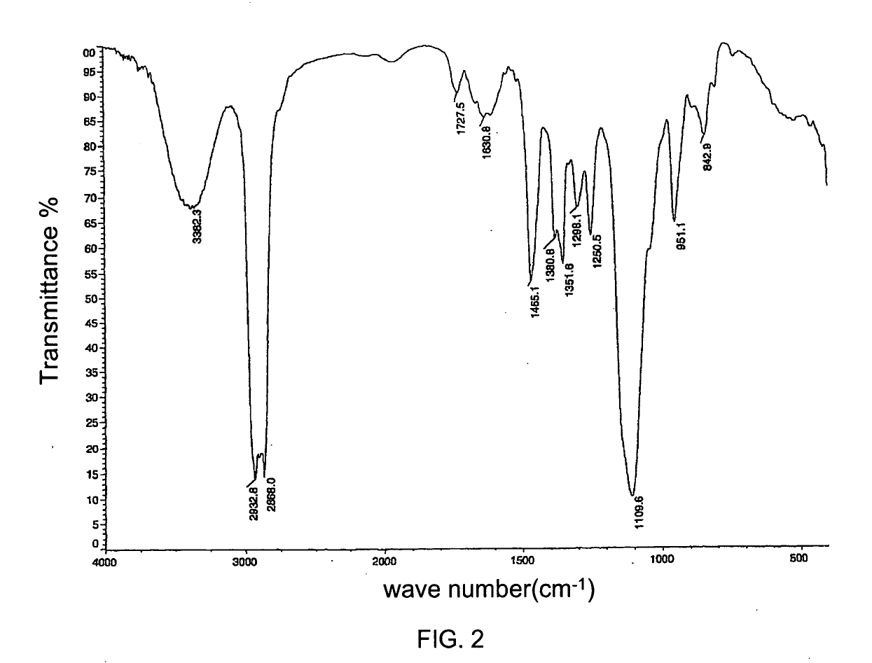

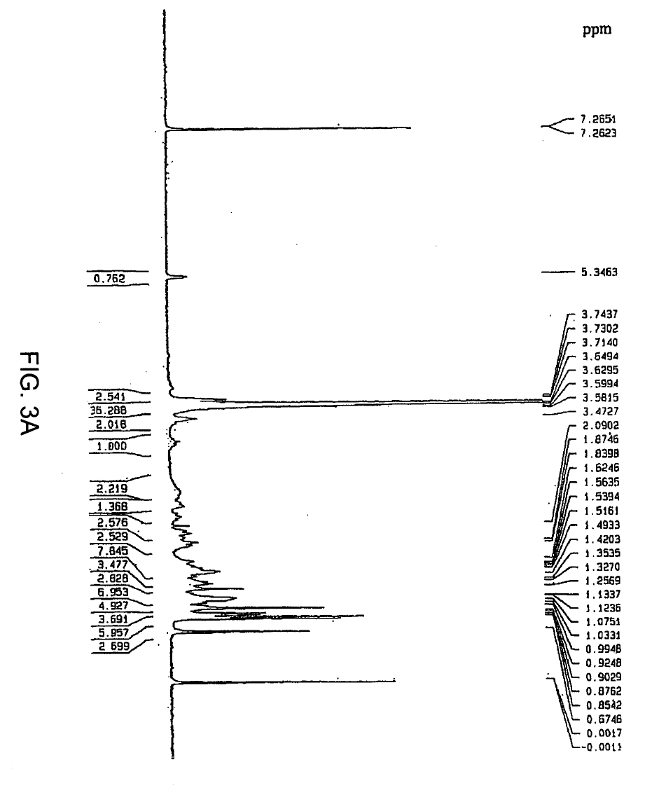

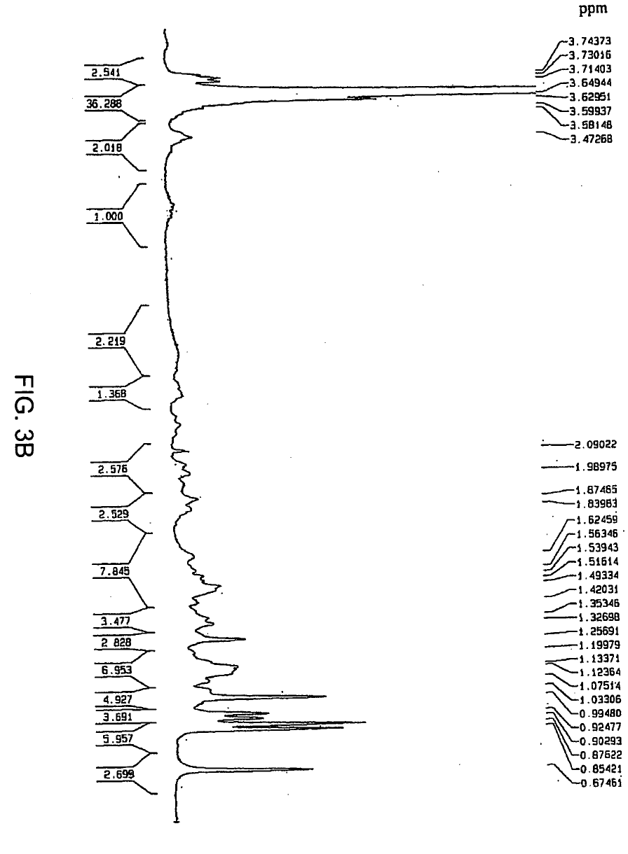

Under the protection of nitrogen, dissolving M1-1 (20 g) in 100 ml anhydrous pyridine, adding p-toluene sulfonyl chloride (8.3 g) into a reaction bottle followed by stirring under room temperature for 16 hours, TLC tracking till finishing the reaction, pouring in 200 ml ice water of 0° C., filtering to collect solid, washing till to neutral, and drying to gain 24 g light yellow solid which is M2-1; under the protection of nitrogen, dissolving M2-1 (20 g) in 400 g PEG-600 (i.e. polyethylene glycol whose molecular weight is 600, and thereinafter is similar) and 300 ml 1,4-dioxane, stirring to react for 2 hours at 80° C., TLC tracking till finishing the reaction, removing dioxane by decompression concentration, adding residues into 400 ml water, extracting with 200 ml ether twice, then washing with 100 ml 5% to 10% sodium carbonate solution for one or two times, washing with water (100 ml) one or two times, drying with anhydrous magnesium sulfate, and removing solvent to gain crude product, wherein column chromatography of the crude product is carried out to gain the cholesterol ether modified by polyethylene glycol showed as the formula, the infrared spectrum thereof is showed in FIG. 2, and mass spectrograms are as shown in FIG. 3A and FIG. 3B (3A and 3B constitute a complete mass spectrogram).

### Example 2 for Preparation

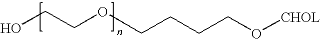

Wherein n is about 5.

Under the protection of nitrogen, dissolving cholesterol (20 g) in 100 ml anhydrous pyridine, adding p-toluene sulfonyl chloride (9.8 g) into a reaction bottle followed by stirring under room temperature for 16 hours, TLC tracking till finishing the reaction, pouring in 200 ml ice water of 0° C., filtering to collect solid, washing till to neutral, and drying to gain 25 g white solid which is cholesterol p-toluenesulfonate; under the protection of nitrogen, dissolving cholesterol p-toluenesulfonate (21.74 g) in 200 ml 1,4-butanediol and 300 ml 1,4-dioxane, stirring to react for 2 hours at 80° C., TLC tracking till finishing the reaction, removing dioxane by decompression concentration, adding residues into 400 ml water, extracting with 200 ml ether twice, then washing with 100 ml 5% to 10% sodium carbonate solution for one or two times, washing with water (100 ml) one or two times, drying with anhydrous magnesium sulfate, and removing solvent to gain crude product, wherein column chromatography of the crude product is carried out to gain the pure: a light yellow solid M1-2.

Under the protection of nitrogen, dissolving M1-2 (20 g) in 100 ml anhydrous pyridine, adding p-toluene sulfonyl chloride (8.3 g) into a reaction bottle followed by stirring under room temperature for 16 hours, TLC tracking till finishing the reaction, pouring in 200 ml ice water of 0° C., filtering to collect solid, washing till to neutral, and drying to gain 24 g light yellow solid which is M2-2; under the protection of nitrogen, dissolving M2-2 (20 g) in 200 g PEG-300 and 300 ml 1,4-dioxane, stirring to react for 2 hours at 80° C., TLC tracking till finishing the reaction, removing dioxane by decompression concentration, adding residues into 400 ml water, extracting with 200 ml ether twice, then washing with 100 ml 5% to 10% sodium carbonate solution for one or two times, washing with water (100 ml) one or two times, drying with anhydrous magnesium sulfate, and removing solvent to gain crude product, wherein column chromatography of the crude product is carried out to gain the cholesterol ether modified by polyethylene glycol, showed as the formula.

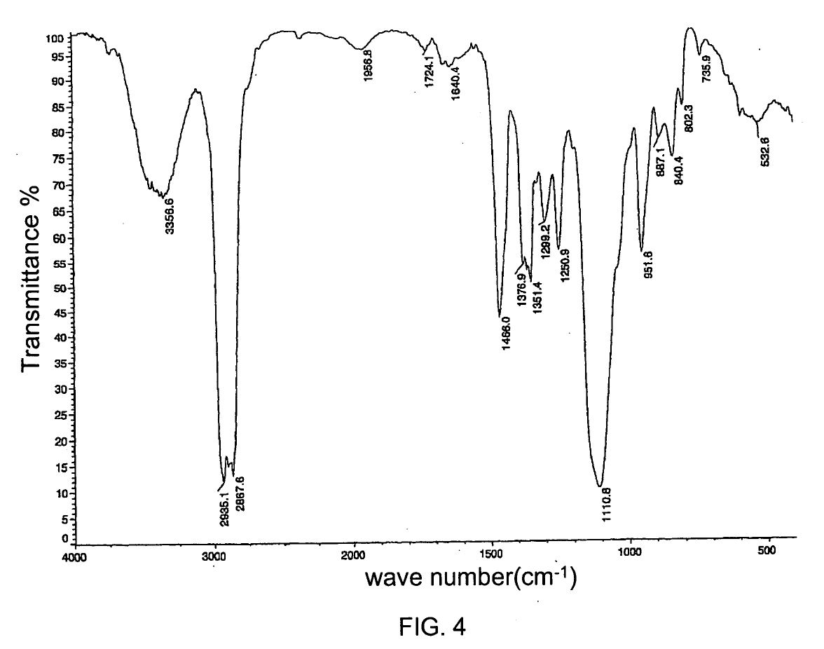

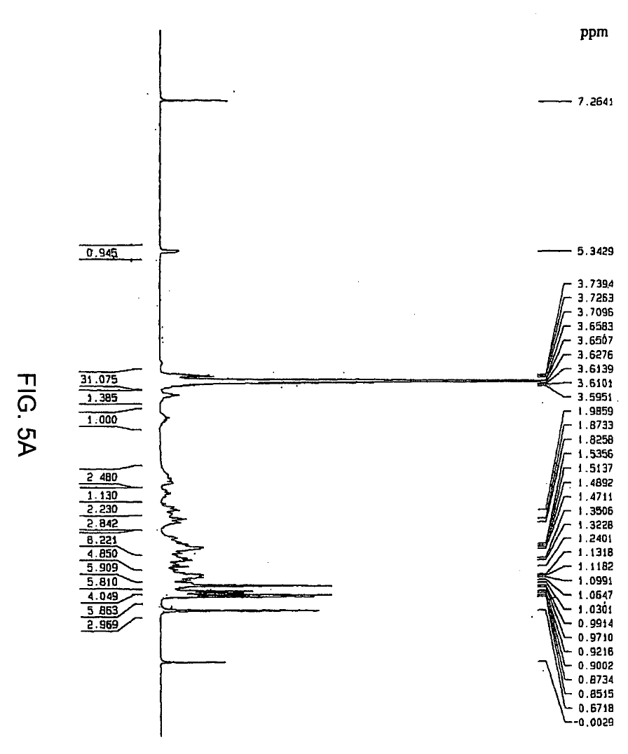

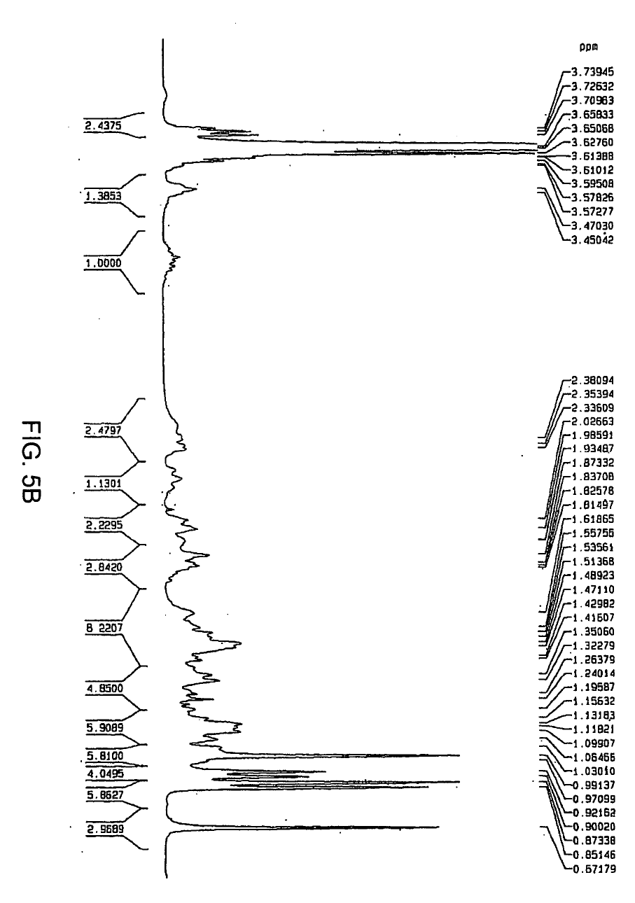

This compound is semi-solid oil-like substance of light yellow, easily dissolved in chloroform, ethyl acetate and ethanol, minimal dissolved in water, infrared spectrum thereof is as shown in FIG. 4, and mass spectrograms are as shown in FIG. 5A and FIG. 5B (FIG. 5A and FIG. 5B constitute a complete mass spectrogram).

### Example 3 for Preparation

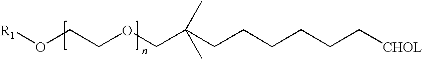

Wherein R₁ is H, and n is about 16.

Under the protection of nitrogen, dissolving cholesterol (20 g) in anhydrous pyridine (100 ml), adding p-toluene sulfonyl chloride (9.8 g) into a reaction bottle followed by stirring under room temperature for 16 hours, TLC tracking till finishing the reaction, pouring in 200 ml ice water of 0° C., filtering to collect solid, washing till to neutral, and drying to gain 25 g white solid which is cholesterol p-toluenesulfonate; under the protection of nitrogen, dissolving cholesterol p-toluenesulfonate (21.74 g) in 200 ml 2,2-dimethyl-1,8-octanediol and 300 ml 1,4-dioxane, stirring to react for 2 hours at 80° C., TLC tracking till finishing the reaction, removing dioxane by decompression concentration, adding residues into 400 ml water, extracting with 200 ml ether twice, then washing with 100 ml 5% to 10% sodium carbonate solution for one or two times, washing with water (100 ml) one or two times, drying with anhydrous magnesium sulfate, and removing solvent to gain crude product, wherein column chromatography of the crude product is carried out to gain the pure: a light yellow solid M1-3.

Under the protection of nitrogen, dissolving M1-3 (20 g) in 100 ml anhydrous pyridine, adding p-toluene sulfonyl chloride (8.3 g) into a reaction bottle followed by stirring under room temperature for 16 hours, TLC tracking till finishing the reaction, pouring in 200 ml ice water of 0° C., filtering to collect solid, washing till to neutral, and drying to gain 24 g light yellow solid which is M2-3; under the protection of nitrogen, dissolving M2-3 (20 g) in 500 g PEG-1000 and 300 ml 1,4-dioxane, stirring to react for 2 hours at 80° C., TLC tracking till finishing the reaction, removing dioxane by decompression concentration, adding residues into 400 ml water, extracting with 200 ml ether twice, then washing with 100 ml 5% to 10% sodium carbonate solution for one or two times, washing with water (100 ml) one or two times, drying with anhydrous magnesium sulfate, and removing solvent to gain crude product, wherein column chromatography of the crude product is carried out to gain the cholesterol ether modified by polyethylene glycol.

### Example 4 for Preparation

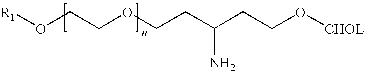

Wherein R₁ is H, and n is about 6.5.

Under the protection of nitrogen, dissolving cholesterol (20 g) in anhydrous pyridine (100 ml), adding p-toluene sulfonyl chloride (9.8 g) into a reaction bottle followed by stirring under room temperature for 16 hours, TLC tracking till finishing the reaction, pouring in 200 ml ice water of 0° C., filtering to collect solid, washing till to neutral, and drying to gain 25 g white solid which is cholesterol p-toluenesulfonate; under the protection of nitrogen, dissolving cholesterol p-toluenesulfonate (21.74 g) in 200 ml 3-amino-1,5-pentanediol and 300 ml 1,4-dioxane, stirring to react for 2 hours at 80° C., TLC tracking till finishing the reaction, removing dioxane by decompression concentration, adding residues into 400 ml water, extracting with 200 ml ether twice, then washing with 100 ml 5% to 10% sodium carbonate solution for one or two times, washing with water (100 ml) one or two times, drying with anhydrous magnesium sulfate, and removing solvent to gain crude product, wherein column chromatography of the crude product is carried out to gain the pure: a light yellow solid M1-4.

Under the protection of nitrogen, dissolving M1-4 (20 g) in 100 ml anhydrous pyridine, adding p-toluene sulfonyl chloride (8.3 g) into a reaction bottle followed by stirring under room temperature for 16 hours, TLC tracking till finishing the reaction, pouring in 200 ml ice water of 0° C., filtering to collect solid, washing till to neutral, and drying to gain 24 g light yellow solid which is M2-4; under the protection of nitrogen, dissolving M2-4 (20 g) in 360 g PEG-400 and 300 ml 1,4-dioxane, stirring to react for 2 hours at 80° C., TLC tracking till finishing the reaction, removing dioxane by decompression concentration, adding residues into 400 ml water, extracting with 200 ml ether twice, then washing with 100 ml 5% to 10% sodium carbonate solution for one or two times, washing with water (100 ml) one or two times, drying with anhydrous magnesium sulfate, and removing solvent to gain crude product, wherein column chromatography of the crude product is carried out to gain the cholesterol ether modified by polyethylene glycol, showed as the formula.

### Example 5 for Preparation

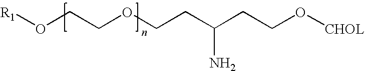

Wherein R₁ is acetyl, and n is about 6.5.

Dissolving 20 g product of examples for preparation 4 in 100 ml dichloromethane, controlling the temperature in ice water below 10 degrees, adding 5 ml acetyl chloride by dropping, stirring for 10 minutes after dropping, then controlling the temperature below 5° C., adding 4 ml pyridine by dropping followed by stirring for 20 minutes, and then pouring in 100 ml water, washing with water till organic phase is into neutral, drying with anhydrous magnesium sulfate, and removing solvent to gain crude product, wherein column chromatography of the crude product is carried out to gain the said compound of cholesterol ether modified by 2-acetyl-polyethylene glycol.

### Example 6 for Preparation

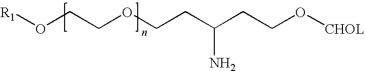

Wherein R₁ is methyl, and n is about 6.5.

Dissolving product of examples for preparation 4 as 0.5 mmol/L in 10 ml water, adding 1 mmol dimethyl sulfate, stirring in ice bath, adding sodium hydroxide solution (1 mol/L, 2 ml) slowly by dropping, reacting for 12 h, TLC tracking the reaction process, adjusting PH value to 2.0 with sulfuric acid after finishing the reaction, filtering by air pumping, wherein silica gel column chromatography of the solid is carried out to gain the compound of cholesterol ether modified by 2-methyl-polyethylene glycol.

### EXAMPLES FOR USE

### Example 1 for Use

### Sub-Nano-Emulsion Formulations

Composing prescriptions 1: 0.01%˜3.0% paclitaxel, 0.01%˜5.0% cosolvent, 0.5%˜06.0% phospholipids, 0.1%˜02.0% PGC (i.e. the product of foregoing examples, hereinafter is the same), 5%˜30% triglyceride, 1.0%˜06.0% glycerol, 1.0%˜6.0% oleic acid, and adding water of injection to 100 ml.

Dissolving 100-500 mg paclitaxel in the appropriate amount of cosolvent (ethanol), dissolving 1.0 g the product PGC of example 1 for preparation (i.e. the product modified by polyethylene glycol 600) in 15 g triglyceride and 0.5 g oleic acid with high-speed stirring to well-mixed at 50-80° C. to form oil phase; evaporating to remove ethanol. Taking 1.0 g egg yolk lecithin, 3 g glycerol, adding recipe quantity of water with high-speed stirring at 50-80° C., fully dispersing to form water phase. Mixing oil phase with water phase with high-speed stirring at 50-80° C. to form pre-emulsion, taking said pre-emulsion, adjusting volume to predetermined recipe quantity with water of injection, transferring to a high-pressure emulsificating machine to emulsify repeatedly till the average particle diameter of emulsion drops is less than or equal to 5 um, bottling in a bottle filled with nitrogen after filtration and sterilization, and sterilizing it to obtain the product.

### Example 2 for Use

### Nano-Emulsion Formulations

Composing prescriptions 1: 0.01%˜2.0% adriamycin, 0.01%˜5.0% adriamycin cosolvent, 0.1%˜3.0% PGC, appropriate assistant surfactant (ethanol, propylene glycol), and adding water of injection to 100 ml.

Dissolving 100-500 mg adriamycin in adriamycin cosolvent (ethanol), adding 2.0 g product of example 2 for preparation (i.e. the product modified by polyethylene glycol 300), 1.0 g propylene glycol and appropriate water at 20-80° C. with stirring to well-mixed, continuing to stir, adding appropriate ethanol by dropping till to semi-clear liquid, and adjusting PH value to 5.0˜7.0, bottling in a bottle filled with nitrogen after filtration and sterilization, and sterilizing it to obtain the product.

### Example 3 for Use

### Micelle

Composing prescriptions 1: 0.01%˜2.0% nimodipine, 0.01%˜5.0% nimodipine cosolvent, 0.1%˜1.0% PGC, appropriate copolymer of polyethylene oxide and poly-amino acid, and adding water of injection to 100 ml.

Dissolving nimodipine, the product PGC of example 2 for preparation (i.e. the product modified by polyethylene glycol 300) and copolymer of polylactic acid-lysine (PLAL) in the appropriate amount of solvent (ethanol), steaming to remove the solvent by decompression, adding recipe quantity of water, stirring uniformly, and high-speed stirring till the average particle diameter of emulsion drops is less than or equal to 5 um, bottling in a bottle filled with nitrogen after filtrating said emulsion, and sterilizing it to obtain the product.

### Example 4 for Use

### Liposome

Composing prescriptions 1: 0.01%˜2.0% vinorelbine, 0.1%˜2.0% PGC, 2.0%˜6.0% lecithin, 5.0%˜15.0% mannitol, and adding water of injection to 100 ml.

Dissolving 100 mg vinorelbine, 2.0 g the product PGC of example 1 for preparation and 4.0 g lecithin in appropriate organic solvent (chloroform), steaming to remove the solvent by decompression, adding recipe quantity of phosphate-buffered salt solution dissolved with sucrose, and completely stirring to form multi-vesicular liposome, transferring to a high-pressure emulsificating machine to emulsify repeatedly till the average particle diameter of emulsions drops is less than or equal to 5 um, bottling in a bottle filled with nitrogen after filtrating said emulsion, and sterilizing it to obtain the product.

### Example 5 for Use

### Solid Lipid Nanoparticles

Composing prescriptions 1: 0.01%˜2.0% hydroxycamptothecin, 0.1%˜3.0% PGC, 1.0%˜6.0% monostearin, 1.0%˜6.0% fatty acid, 2.0%˜6.0% lecithin, 5-20% sucrose, 2.0%˜6.0% mannitol, and adding water of injection to 30 ml.

a) Heating to melt 2.0 g the recipe quantity of monostearin, 1.0 g fatty acid, 2.0 g lecithin, 1.5 g the product PGC of example 4 for preparation and 100 mg hydroxycamptothecin to form oil phase, adding 15 g sucrose into recipe quantity of water (PH 10-11) of injection, fully dissolving to form water phase, mixing two phases by high-speed stirring to form pre-emulsion, adjusting PH value to 4.5-8.0, then repeating to high pressure homogenize and emulsify the pre-emulsion system by a high pressure homogenizer, until the particle diameter meet the required regulation.

b) Filtering said pre-emulsion, freezing to dry and sealed with filled nitrogen, so as to obtain the product.

### Example 6 for Use

### Orally Self-Emulsifying Drug Delivery System

Composing prescriptions 1: 0.1%˜10.0% megestrol acetate, 2.0%˜10.0% PGC, 20.0%˜40.0% capric acid, 5.0%˜20.0% Fluoronic F68, and 10.0%˜20.0% polyoxyethylene castor oil.

Well-mixing the recipe quantity of megestrol acetate, PGC, the product of example 3 for preparation, capric acid, Fluoronic F68 and polyoxyethylene castor oil with repeated grinding, heating to melt it to obtain self-emulsifying drug delivery system for orally.

This system can be diluted into oral emulsion according to practical methods, and also can be pressed into soft gelatin capsules or bottled into liquid hard capsule.

### Example 7 for Use

### Vascular Irritation Test

Experimental drug: preparing in accordance with the method provided by examples 1, 2, 4 and 5 for use, totally 4 types; hydroxycamptothecin injection solution (purchased from the market), diluting into 5% solution with 0.9% sodium chloride injection solution during testing.

Experimental animals: healthy rabbits, weight 2.3˜2.4 kg.

Experimental methods: taking 10 healthy rabbits, equally divided between male and female. Dividing into the control group of 0.9% sodium chloride injection solution, the group of hydroxycamptothecin injection solution and the group of examples (1, 2, 4 and 5) for use according to the weight and gender, two rabbits in each group, 10 ml/kg intravenous drip according to clinical drug concentration is carried out at the left edge of rabbit ears, the injection speed was 1 ml per minute, one times daily for 7 days. Intravenous drip of 0.9% sodium chloride injection solution is carried out for the control group with the same way. Except for observing the status of local administration while administration and after administration, cutting the auricle of administration ear side after the final intravenous drip, after conventional fixing, cutting specimen of 0.5 cm width at each 1 cm interval away from proximal end 1 cm of intravenous drip, totally three specimens. Sections staining, observing the pathological through a microscope, results are shown in the following table:

Vascular irritation test

| | | | Whether |
|---|---|---|---|
| | | | inflammatory |
| | Vasodilation | | cell |
| | of | | infiltration |
| Item | rabbit's ear | Inflamed | exists |
| control group of 0.9% sodium | − | − | − |
| chloride injection solution | | | |
| hydroxycamptothecin | ++ | ++ | + |
| injection solution | | | |
| Example 1 for use | − | − | − |
| Example 2 for use | − | − | − |
| Example 4 for use | − | − | − |
| Example 5 for use | − | − | − |

Remarks:
“++” represents serious,
“+” represents slight,
“−” represents no

The results showed that the preparation prepared by the present invention has the advantage of slight stimulation.

### Example 8 for Use

### Preliminary Pharmacokinetic Study of PGC Used for Manufacturing Voriconazole Nano-Preparation

12 healthy SD rats, the number of male and female are each half, weight about 220 g, randomly divided into 2 groups, food and drinking freely. One group, injecting 36 mg/kg voriconazole nano-preparation solution (delivery volume 9 ml/kg) using PGC of example 2 (i.e. sterol modified by PEG-300) as excipient and manufactured according to conventional emulsion technology at tail vein of mouse slowly; another group, injecting 36 mg/kg voriconazole nano-preparation solution (delivery volume 9 ml/kg) (manufactured according to conventional emulsion technology) without using PGC as excipient and manufactured according to high-pressure emulsificating at tail vein of mouse slowly. Collecting plasma separately at 5, 10, 30, 60 minutes before injection, and 2, 4, 6, 8, 10, 12, 24 hours after injection, followed by being determined by high performance liquid chromatography.

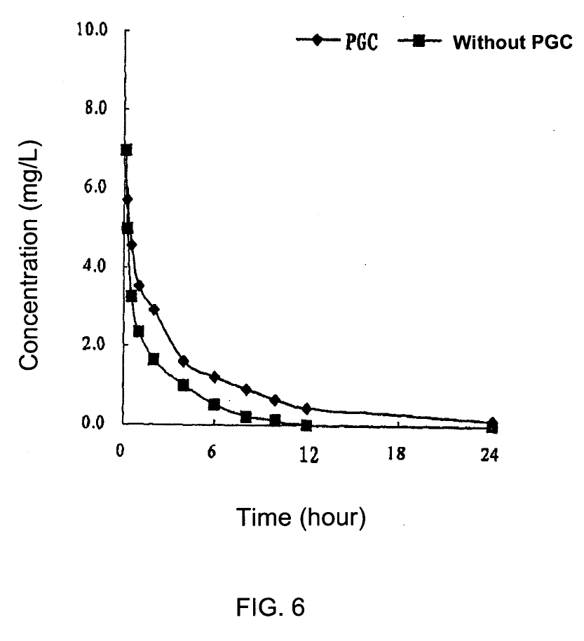

The average plasma drug concentration-time curve of voriconazole after the intravenous injection of two kinds of 36 mg/kg voriconazole in rats is shown in FIG. 6, wherein “PGC” presents for voriconazole nano-preparation using PGC as excipient, “without PGC” presents for voriconazole nano-preparation without using PGC as excipient.

As shown in the figures: after administrating voriconazole nano-preparation using PGC as excipient, plasma drug concentration declines slowly and voriconazole can still be detected after 24 hours, indicating that this preparation has a certain slow release characteristics, for example in intravenous drip, the slow release can be achieved by one time daily administration, so that the times of administration could be reduced and the patient's compliance could increased; however, after administrating voriconazole nano-preparation without using PGC as excipient and manufactured according high-pressure emulsificating, plasma drug concentration declines rapidly and closes to the concentration of limit quantification after 12 hours, voriconazole can almost not be detected, indicating that pharmacokinetic thereof is poor.

The inventor of the present invention thinks that voriconazole has a larger molecular structure including 3 large ring structures, thus it is difficult to be completely encapsulated in the nano-ball of phospholipids without leakage or precipitation. However, a new kind of excipient PGC of the present invention is used and can package the drugs effectively and encapsulation efficiency is up to 98%, consequently the elimination of drugs in animals is delayed and has a long cycle effective time, and the effect of slow release is obvious, indicating that the pharmacokinetic is good. Simultaneously, the characters of preparation are translucent white liquid, blue opalescent and have well characters.

Overall, nano-preparation manufactured by PGC is better than ordinary emulsion, injection solution and freeze-dried powder injection and has broad market prospects and competitiveness.

What is claimed is:

1. A sterol modified by polyethylene glycol, wherein the formula thereof is: 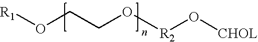
wherein CHOL is a sterol compound;
R₁ is H, straight or branched alkyl, alkenyl or alkynyl, or straight or branched alcohol, ketone, ether, ester, carboxyl or salt thereof, amine or amide group;
R₂ is straight or branched alkyl, alkenyl or alkynyl, or ketone, ether, ester, amine or amide group, wherein R₂ is not represented by —CH₂CH₂—;
n is an integer from 1 to 40.

2. The sterol modified by polyethylene glycol according to claim 1, wherein said CHOL is cholesterol, 7-dehydrocholesterol, ergosterol, vitamin D3 or vitamin D2; said CHOL is preferably cholesterol.

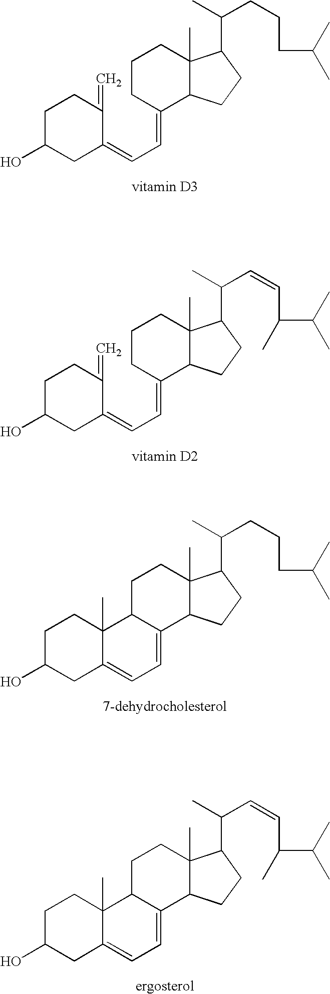

3. The sterol modified by polyethylene glycol according to claim 1, wherein said R₁ is H, —CH₃, —CH₂CH₃, n-propyl or isopropyl, n-butyl, isobutyl, sec-butyl, tert-butyl, —CH₂CH(CH₂CH₂)ₘCH₃, —CH═CHCH₃, —CH₂C═CH₂, —CH₂OH, —CH₂COCH₃, —CH₂O(CH₂CH₂)ₘCH₃, —CH₂COOH, —CH₂CH₂NH₂ or —COCH₃; wherein m is an integer from 1 to 30; R₁ is preferably —H, —CH₃, or —COCH₃.

4. The sterol modified by polyethylene glycol according to claim 1, wherein R₂ is —CH₂—, —CH₂(CH₂)ₘCH₂—, —CH₂C(CH₃)₂—CH₂—(CH₂)ₘ—, —CH₂CH₂(CH₂CH₂)ₘCH₂—, —CH═CHCH₂—, —CH₂C═CH—, —CH₂OCH₂—, —CH₂COCH₂—, —CH₂O(CH₂CH₂)ₘCH₂—, —CH₂COOCH₂—; m is an integer from 1 to 30; wherein each of said groups is optionally substituted by one or more amino group; R₂ is preferably —CH₂CH₂—, —CH₂(CH₂)ₘCH₂—, —CH₂C(CH₃)₂—CH₂—(CH₂)ₘ—; m is an integer from 1 to 12, preferably from 1 to 8; especially from 1 to 6, wherein each of said groups is optionally substituted by one or more amino group.

5. The sterol modified by polyethylene glycol according to claim 1, wherein n is from 3 to 20.

6. A method for preparing sterol modified by polyethylene glycol of claim 1, comprising:
(a) synthesizing a steryl p-toluenesulfonate;
(b) reacting steryl p-toluenesulfonate synthesized by step (a) with a kind of OH—R₂—OH;
(c) reacting a product of step (b) with a kind of polyethylene glycol;
(d) optionally, introducing one R₁ group into a product of step (c).

7. The method according to claim 6, wherein said product of step (b) is preferably activated by p-toluene sulfonyl halide, and then reacted with polyethylene glycol, wherein solvent is preferably 1,4-dioxane.

8. The method according to claim 6, wherein in said step (a), sterol is reacted with p-toluene sulfonyl halide to form steryl p-toluenesulfonate, wherein the halide is preferably fluoride, chloride or bromide, especially chloride.

9. The method according to claim 6, wherein HO—R₂—OH used in said step (b) is HO—CH₂(CH₂)ₘCH₂—OH, HO—CH₂C(CH₃)₂—CH₂—(CH₂)ₘ—OH, wherein m is an integer from 1 to 30, preferably from 1 to 12, especially from 1 to 8, particularly from 1 to 6, wherein each group represented by R₂ is optionally substituted by one or more amino group.

10. A use of sterol according to claim 1, used as a surfactant for manufacturing aqueous dispersion system, such as nano-emulsion, liposome; used as one component of a carrier for manufacturing micelle or solid lipid nanoparticles which can be dispersed in water; used as one component of a long-circulation nano-carrier component, temperature-sensitive or PH-sensitive nano-carrier component; used as a cosmetic excipient; and applied to hypodermic for smoothing away wrinkles.
